(19) United States
(12) Patent Application Publication
(10) Pub. No.: US 2021/0340177 A1
(43) Pub. Date: Nov. 4, 2021

(54) BIOACTIVE PEPTIDES HAVING HIGH BINDING AFFINITY TO HUMAN MUSCULAR NICOTINIC ACETYLCHOLINE RECEPTOR

(71) Applicant: L'OREAL, Paris (FR)

(72) Inventors: Rodrigo DE VECCHI, Rio de Janeiro (BR); Lionel BRETON, Rio de Janeiro - RJ (BR); Leonardo de Azevedo CALDERON, Rio de Janeiro - RJ (BR); Fernando ZANCHI, Rio de Janeiro - RJ (BR); Aleff Ferreira FRANCISCO, Rio de Janeiro - RJ (BR)

(21) Appl. No.: 17/051,897

(22) PCT Filed: Apr. 30, 2020
(86) PCT No.: PCT/BR2018/050141

§ 371 Date: Oct. 30, 2020

(51) Int. Cl. C07K 5/10; A61K 8/64; A61Q 19/08; A61P 21/02

(52) U.S. Cl. CPC C07K 5/10; A61P 21/02; A61Q 19/08; A61K 8/64

(57) ABSTRACT

Two synthetic tetrapeptides having a high binding affinity to human muscular nicotinic acetylcholine receptor (HmnAChR) are provided. A composition for inhibiting contraction of muscle cells is also provided. The composition comprises an effective amount of one of these two peptides. Also provided is a method for inhibiting contraction of muscle cells, which method comprises treating muscle cells with an effective amount of the composition. Further provided is a method for inducing proliferation of epidermal cells, which method comprises treating the epidermal cells with an effective amount of the composition. The epidermal cells may be human epidermal keratinocytes (HEKs).

### FIELD OF THE INVENTION

The invention relates to bioactive peptides having high binding affinity to human muscular nicotinic acetylcholine receptor (HmnAChR) and uses thereof.

### BACKGROUND OF THE INVENTION

Peptides derived from natural peptide precursors isolated from, for example, snake venom, are widely used for various therapeutic or cosmetic purposes. A well-known example is captopril, whose natural peptide precursor is isolated from Bothrops jararaca snake venom. Captopril is a peptide-based drug that inhibits the angiotensin-converting enzyme, producing an antihypertensive effect. Other useful peptides prepared from natural peptide precursors include natriuretic peptides, bradykinin-potentiating peptides and sarafotoxins. Low mass proteins such as crotamine, disintegrins and three-Finger toxins are derived from snake venoms.

Some commercial peptide products contain synthetic peptides derived from natural peptides isolated from snake venom. For example, SYN®-AKE (DSM) is an effective wrinkle-smoothing compound based on a synthetic tripeptide, i.e., dipeptide diaminobutyroyl benzylamide diacetate, that mimics the activity of Waglerin 1, a polypeptide found in the venom of the Temple Viper, Tropidolaemus wagleri. SYN®-AKE acts at the postsynaptic membrane and is a reversible antagonist of the muscular nicotinic acetylcholine receptor (mnAChR). Upon binding to the mnAChR by SYN®-AKE, Na⁺ uptake is blocked at the postsynaptic membrane and muscle cell contraction is attenuated. SYN®-AKE is able to reduce signal transmission between nerves and works in a manner similar to Botox to relax muscles.

There remains a need for bioactive peptides, especially low mass peptides, having a high binding affinity to human muscular nicotinic acetylcholine receptor (HmnAChR) as effective muscle relaxants.

### SUMMARY OF THE INVENTION

The present invention relates to synthetic bioactive tetra-peptides and uses thereof.

A peptide consisting of an amino acid sequence of SEQ ID NO: 1 or 2 is provided. The peptide may consist of the amino acid sequence of SEQ ID NO: 1. The peptide may consist of the amino acid sequence of SEQ ID NO: 2.

A composition for inhibiting contraction of muscle cells is provided. The composition comprises an effective amount of a peptide consisting of an amino acid sequence of SEQ ID NO: 1 or 2. The peptide may consist of the amino acid sequence of SEQ ID NO: 1. The peptide may consist of the amino acid sequence of SEQ ID NO: 2. The peptide may be present at a concentration of 0.01-500 μM. The inhibition may be irreversible.

A method for inhibiting contraction of muscle cells is provided. The method comprises treating muscle cells with an effective amount of a composition. The composition comprises a peptide. The peptide consists of an amino acid sequence of SEQ ID NO: 1 or 2. As a result, contraction of the muscle cells is inhibited. The peptide may consist of the amino acid sequence of SEQ ID NO: 1. The peptide may consist of the amino acid sequence of SEQ ID NO: 2. The composition may comprise the peptide at a concentration of 0.01-500 μM. The muscle cells may be treated with the peptide for at least 5 hours. The muscle cells may be treated with the composition for at least 24 hours. The inhibition may be irreversible. The muscle cells may be in a subject.

A method for reducing appearance of wrinkles on skin of a subject is provided. The method comprises treating skin having appearance of wrinkles with an effective amount of a composition. The composition comprises a peptide. The peptide consists of an amino acid sequence of SEQ ID NO: 1 or 2. As a result, the appearance of wrinkles on the skin is reduced. The peptide may consist of the amino acid sequence of SEQ ID NO: 1. The peptide may consist of the amino acid sequence of SEQ ID NO: 2.

A method for inducing proliferation of epidermal cells is provided. The method comprises treating the epidermal cells with an effective amount of a composition. The composition comprises a peptide. The peptide consists of an amino acid sequence of SEQ ID NO: 1 or 2. As a result, proliferation of the epidermal cells is induced. The peptide may consist of the amino acid sequence of SEQ ID NO: 1. The peptide may consist of the amino acid sequence of SEQ ID NO: 2. The epidermal cells may be human epidermal keratinocytes (HEKs).

### BRIEF DESCRIPTION OF THE DRAWINGS

FIGS. 1A, 1B and 1C show (A) acetylcholine, (B) SYN®-AKE, and (C) peptide KKYK (SEQ ID NO: 1) in the binding site between chains E and A of human muscular nicotinic acetylcholine receptor (HmnAChR).

FIG. 2 is a peptide interaction graph showing binding energy (kcal/mol) of peptides over runs of genetic algorithm.

FIGS. 3A and 3B show a non-cytoxic effect of peptide Lys-Lys-Tyr-Lys (KKYK; SEQ ID NO: 1) or peptide Lys-Trp-Lys-Lys (KWKK; SEQ ID NO: 2) on human epidermal keratinocytes (HEKs) in in vitro cultures.

FIGS. 4A and 4B show inhibition of contraction of muscle cells by peptide V0083091A (KKYK; SEQ ID NO: 1) and peptide V0083092A (KWKK; SEQ ID NO: 2) at 0, 0.01, 0.1, 1, 5, 10, 50, 100 or 500 μM.

### DETAILED DESCRIPTION OF THE INVENTION

The present invention provides synthetic low mass bioactive peptides and uses thereof in the cosmetic field or other fields. The peptide may be used, for example, as muscle relaxants in anesthetic, anti-wrinkle and anti-aging products. The invention was made based on the discovery that two synthetic bioactive tetra-peptides KKYK (SEQ ID NO: 1) and KWKK (SEQ ID NO: 2) having a strong selective affinity to human muscular nicotinic acetylcholine receptor (HmnAChR) are safe and effective in inhibiting contraction of muscle cells.

The term “peptide” used herein refers to a compound having two or more amino acids linked in a chain, with or without a branched chain. Any amino acid in the peptide may have one or more post-translation modifications. The term “low mass peptide” used herein refers to a peptide having no more than 5, 10, 50 or 100 amino acids. In one embodiment, the peptide is a tetra-peptide having four amino acids linked in a straight chain without a branched chain.

The term “an effective amount” refers to an amount of a peptide or a composition comprising a peptide according to this invention that is required to achieve a stated goal. The effective amount may be selected to inhibit contraction of muscle cells, inducing proliferation of cells such as epidermal cells (e.g., HEKs), or reducing appearance of wrinkles on skin of a subject. The effective amount may vary depending on the nature of the peptide or the composition, the type of the target cells, treatment time, and the stated goal. A specific effective amount for a given peptide or a given composition may generally be set by the judgement of a technician.

Two synthetic peptides are provided. One peptide consists of an amino acid sequence of KKYK (SEQ ID NO: 1). The other peptide consists of an amino acid sequence of KWKK (SEQ ID NO: 2). These peptides have a high binding affinity to human muscular nicotinic acetylcholine receptor (HmnAChR). Other properties of these peptides are shown in the examples.

A composition for inhibiting contraction of muscle cells is provided. The composition comprises an effective amount of a peptide. The peptide consists of an amino acid sequence of KKYK (SEQ ID NO: 1) or KWKK (SEQ ID NO: 2). In one embodiment, the peptide consists of the amino acid sequence of SEQ ID NO: 1. In another embodiment, the peptide consists of the amino acid sequence of SEQ ID NO: 2. The composition may comprise the peptide at a concentration of about 0.01-500 μM, about 0.1-100 μM, about 1-50 μM or about 1-10 ∥M, for example, 1 or 10 μM. The inhibition of the contraction of the muscle cells may be irreversible.

The composition may be a cosmetic composition and further comprises a cosmetically acceptable carrier. The composition may be a pharmaceutical composition and comprises a pharmaceutically acceptable carrier.

A method for inhibiting contraction of muscle cells is provided. The method comprises treating muscle cells with an effective amount of a composition. The composition comprises a peptide. The peptide consists of an amino acid sequence of KKYK (SEQ ID NO: 1) or KWKK (SEQ ID NO: 2). As a result, contraction of the muscle cells is inhibited. The inhibition may be irreversible. In one embodiment, the peptide consists of the amino acid sequence of SEQ ID NO: 1. In another embodiment, the peptide consists of the amino acid sequence of SEQ ID NO: 2.

According to the inhibition method, the muscle cells may be treated for at least about 0.5, 1, 2, 5, 10, 12, 18, 24 or 48 hours, for example, at least about 5 or 24 hours. The contraction of the muscle cells may be inhibited by at least about 5%, 10%, 20%, 30%, 40%, 50%, 60%, 70%, 80%, 90, 95% or 99% as compared with that before the treatment.

In some embodiments, the muscle cells are in a muscle of a subject. Upon treatment of the muscle cells with a composition comprising peptide KKYK (SEQ ID NO: 1) or KWKK (SEQ ID NO: 2), the muscle may be relaxed.

A method for reducing appearance of wrinkles on skin of a subject is provided. The method comprises treating skin having appearance of wrinkles with an effective amount of a composition. The composition comprises a peptide. The peptide consists of an amino acid sequence of SEQ ID NO: 1 or 2. As a result, the appearance of wrinkles on the skin is reduced. In one embodiment, the peptide consists of the amino acid sequence of SEQ ID NO: 1. In another embodiment, the peptide consists of the amino acid sequence of SEQ ID NO: 2. The skin may be treated at least once, twice or three times daily and/or for at least about 7, 14, 21, 28, 60 or 90 days.

A method for inducing proliferation of epidermal cells is provided. The method comprises treating the epidermal cells with an effective amount of a composition. The composition comprises a peptide. The peptide consists of an amino acid sequence of SEQ ID NO: 1 or 2. As a result, proliferation of the epidermal cells is induced. In one embodiment, the peptide consists of the amino acid sequence of SEQ ID NO: 1. In another embodiment, the peptide consists of the amino acid sequence of SEQ ID NO: 2.

The epidermal cells may be human epidermal keratinocytes (HEKs). The epidermal cells may be treated with the composition once or more times, each time for at least about 0.5, 1, 2, 5, 10, 12, 18, 24 or 48 hours, for example, at least 24 hours.

### EXAMPLE 1

### Peptide V0083091A

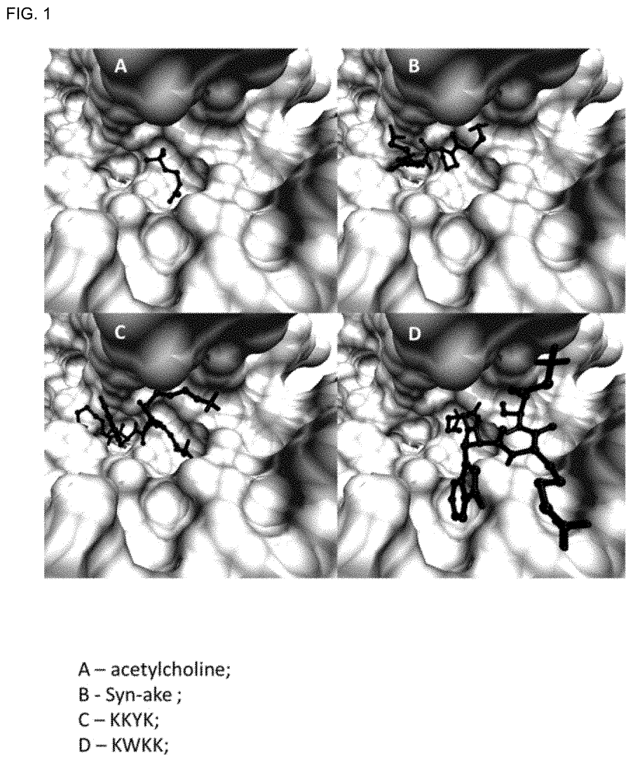

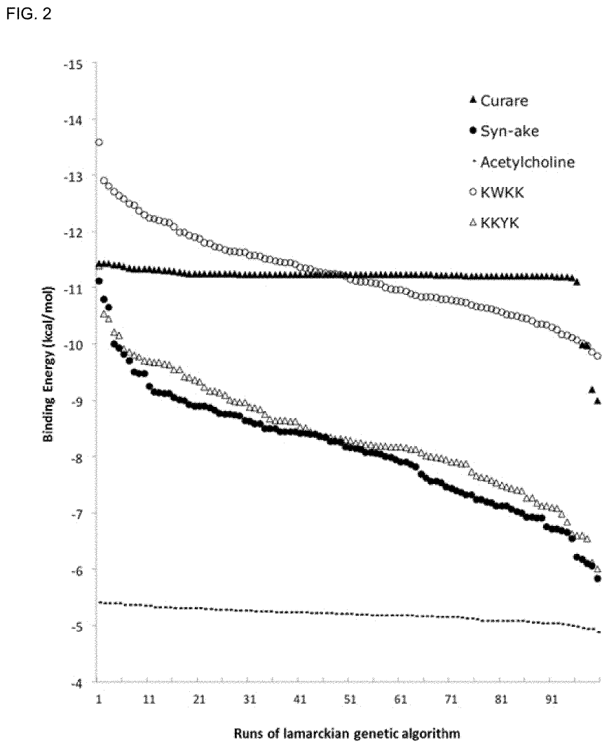

Derived from phospholipases (UniprotKB Q8UVU7) of Bothrops godmani, low mass peptide V0083091A was synthesized to have an enhanced selective affinity to human muscular nicotinic acetylcholine receptor (HmnAChR) (FIG. 1). Peptide V0083091A consists of an amino acid sequence of KKYK (SEQ ID NO: 1) (Table 1), and showed HmnAChR a binding affinity similar to that of SYN®-AKE (Table 2; FIG. 2).

TABLE 1
Physicochemical properties of peptide V0083091A

| | | Physicochemical properties | | | | |
|---|---|---|---|---|---|---|
| | | Molec- | | Iso- | | |
| | Number | ular | Extinction | electric | Net | |
| | of | weight | coefficient | point | charge | Aqueous |
| Sequence | residues | (g/mol) | (M⁻¹cm⁻¹) | (pH) | at pH 7 | solubility |
| KKYK | 4 | 565.71 | 1280 | 10.55 | 3.0 | Good |
| SYN ®- | 3 | 375.47 | — | — | 2.0 | Good |
| AKE | | | | | | |

TABLE 2
Binding energy of peptide V0083091A

| | Binding Energy (kcal/mol) | | | | |
|---|---|---|---|---|---|
| Primary | | Top 10 | | All 100 | |
| Structure | Best Result | Average | S.D. | Average | S.D. |
| KKYK | −11.39 | −10.18 | 0.52 | −8.39 | 1.03 |
| SYN ®-AKE | −11.12 | −10.05 | 0.57 | −8.11 | 1.05 |

S.D. Standard deviation

### EXAMPLE 2

### Peptide V0083092A

Low mass peptide V0083092A was synthesized to have an enhanced selective affinity to human muscular nicotinic acetylcholine receptor (HmnAChR). Peptide V0083092A consists of an amino acid sequence of KWKK (SEQ ID NO: 2) and showed HmnAChR a binding affinity similar to that of SYN®-AKE (Table 3).

TABLE 3
Binding energy and physicochemical
properties of peptide V0083092A

| | | | | (Best | Net | |
|---|---|---|---|---|---|---|
| | Best | | | Result − | charge | Aqueous |
| Peptide | Result | Average | S.D. | Average) | at pH 7 | solubility |
| KWKK | −13.58 | −11.22 | 0.75 | −2.35 | 3.0 | Good |
| SYN ®- | −11.12 | −8.11 | 1.05 | −3.01 | 2.0 | Good |
| AKE | | | | | | |

S.D. Standard deviation

### EXAMPLE 3

### Safety of peptides V0083091A and V0083092A

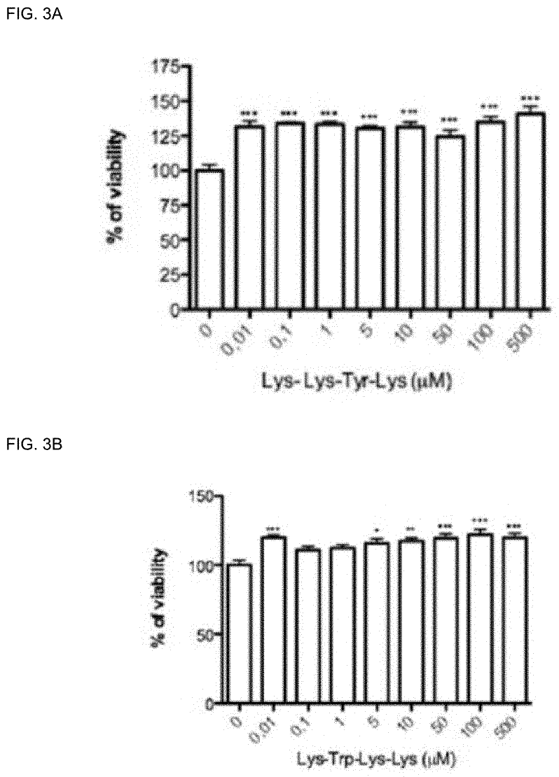

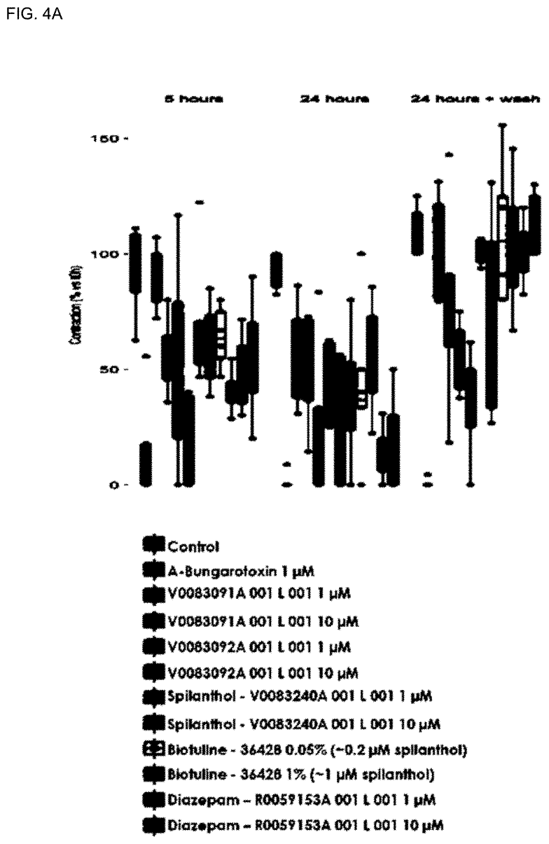

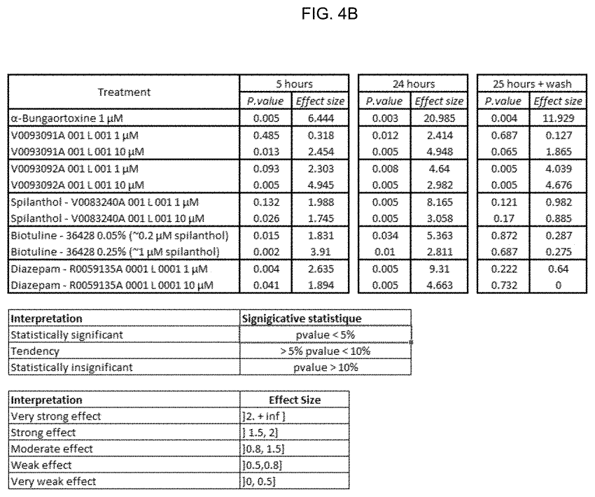

The safety of peptides V0083091A (KKYK; SEQ ID NO: 1) and V0083092A (KWKK; SEQ ID NO: 2) was studied. Human epidermal keratinocytes (HEKs) in in vitro cultures were treated with either peptide. An MTT assay was carried out 24 hours after the treatment and the MTT reading was obtained at 570 nm for assessing cell metabolic activity. Each peptide showed a non-cytotoxic effect on viability of the HEKs (FIG. 3A) and induced proliferation of the HEKs (FIG. 3B). Other peptides (Table 4) were also found to be non-cytotoxic and have high specific binding affinity to HmnAChR as compared to SYN®-AKE. These results suggest an anti-wrinkle benefit related to epidermal regeneration.

TABLE 4
Other peptides

| | SEQ ID | | SEQ ID | | SEQ ID |
|---|---|---|---|---|---|
| Peptide | NO | Peptide | NO | Peptide | NO |
| KYWL | 3 | GKIP | 29 | NYKI | 55 |
| KYWF | 4 | YLQK | 30 | FCKK | 56 |
| YPAK | 5 | WFYG | 31 | KKKW | 57 |
| NKKY | 6 | CYKK | 32 | YWFY | 58 |
| KYKI | 7 | YSSY | 33 | KKYK | 59 |
| NYKI | 8 | FYPA | 34 | KFKK | 60 |
| FCKK | 9 | TYNK | 35 | KKKY | 61 |
| YWFY | 10 | KKLT | 36 | KYKK | 62 |
| FYPAK | 11 | CKKP | 37 | YKKK | 63 |
| WFYP | 12 | FFCK | 38 | FKKK | 64 |
| YNKK | 13 | YGAK | 39 | KKKI | 65 |
| KALAI | 14 | KNNY | 40 | KKFK | 66 |
| YGCY | 15 | KALA | 41 | KKIK | 67 |
| CCYKK | 16 | KFFCK | 42 | KKKF | 68 |
| WFYPA | 17 | RNYL | 43 | KKLK | 69 |
| GKVF | 18 | YLKP | 44 | KLKK | 70 |
| NKYWF | 19 | QLGK | 45 | KKKS | 71 |
| KFFC | 20 | LKPF | 46 | LKKK | 72 |
| KYWFY | 21 | KNYK | 47 | IKKK | 73 |
| IPSP | 22 | WKKK | 48 | KIKK | 74 |
| KVFL | 23 | KYWL | 49 | KSKK | 75 |
| YWFYP | 24 | KKWK | 50 | KKSK | 76 |
| WKTYW | 25 | KYWF | 51 | KKPK | 77 |
| NRNY | 26 | YPAK | 52 | KKKL | 78 |
| FANL | 27 | NKKY | 53 | KPKK | 79 |
| FCKKP | 28 | KYKI | 54 | PKKK | 80 |

### EXAMPLE 4

### Efficacy of Peptides V0083091A and V0083092A

The efficacy of peptides V0083091A (KKYK; SEQ ID NO: 1) and V0083092A (KWKK; SEQ ID NO: 2) was studied. Muscle cells were incubated or treated without (control) or with compounds, for example, peptides V0083091A and V0083092A, and contraction of the muscle cells was evaluated after incubation for 5 hours, after incubation for 24 hours, or after incubation for 24 hours followed by a washing step.

When tested at 10 μM, both peptides V0083091A and V0083092A inhibited contraction of muscle cells significantly (p value <5%) and very strongly (effect size>2) after incubation for 5 or 24 hours. This inhibition effect was always visible and slightly more marked after 24 hours. When tested at 1 μM, both peptides showed a much less inhibition effect on muscle cell contraction and observable inhibition effect after incubation for 24 hours. After the washing step carried out at the end of the incubation for 24 hours, no clear recovery of the contraction power of the muscle cells was observed. These results suggest that these peptides V0083091A and V0083092A are high stable and have a high affinity for the neuromuscular junction with an irreversible inhibition effect on contraction of muscle cells. The other peptides (Table 4) were also tested, but peptides V0083091A and V0083092A worked the best.

When tested at 1 μM or 10 μM, spilanthol (V0083240A) inhibited contraction of the muscle cells significantly (p value<5%) and very strongly (effect size of 1.5-2) at 5 or 24 hours after incubation. Interestingly, this inhibitory effect was lost after the washing step at the end of the incubation. These results show that spilanthol does not bind covalently and that the inhibitory effect is reversible, which is of great interest at the regulatory/safety level. A similar effect was observed with Biotulin (36428) when tested at 0.05% (˜0.2 μM) and 0.25% (˜1 μM). Even though the effect of spilanthol is more moderate, compared to what can be observed with a-bungarotoxin or peptides, the effect was observable at low concentrations of the active ingredient.

When tested at 1 and 10 μM, diazepam (R0059153A) showed the same profile as spilanthol, however, in this case, the effect observed is slightly larger.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

Although the invention is illustrated and described herein with reference to specific embodiments, the invention is not intended to be limited to the details shown. Rather, various modifications may be made in the details within the scope and range of equivalents of the claims without departing from the invention.

1. A peptide consisting of an amino acid sequence of SEQ ID NO: 1 or 2.

2. The peptide of claim 1, wherein the peptide consists of the amino acid sequence of SEQ ID NO: 1.

3. The peptide of claim 1, wherein the peptide consists of the amino acid sequence of SEQ ID NO: 2.

4. A composition for inhibiting contraction of muscle cells, comprising an effective amount of a peptide consisting of an amino acid sequence of SEQ ID NO: 1 or 2.

5. The composition of claim 4, wherein the peptide consists of the amino acid sequence of SEQ ID NO: 1.

6. The composition of claim 4, wherein the peptide consists of the amino acid sequence of SEQ ID NO: 2.

7. The composition of claim 4, wherein the peptide is present at a concentration of 0.01-500 μM.

8. The composition of claim 4, wherein the inhibition is irreversible.

9. A method for inhibiting contraction of muscle cells, comprising treating muscle cells with an effective amount of a composition, wherein the composition comprises a peptide, wherein the peptide consists of an amino acid sequence of SEQ ID NO: 1 or 2, whereby contraction of the muscle cells is inhibited.

10. The method of claim 9, wherein the peptide consists of the amino acid sequence of SEQ ID NO: 1.

11. The method of claim 9, wherein the peptide consists of the amino acid sequence of SEQ ID NO: 2.

12. The method of claim 9, wherein the composition comprises the peptide at a concentration of 0.01-500 μM.

13. The method of claim 9, wherein the muscle cells are treated with the peptide for at least 5 hours.

14. The method of claim 9, wherein the muscle cells are treated with the composition for at least 24 hours.

15. The method of claim 9, wherein the inhibition is irreversible.

16. A method for inducing proliferation of epidermal cells, comprising treating epidermal cells with an effective amount of a composition, wherein the composition comprises a peptide, wherein the peptide consists of an amino acid sequence of SEQ ID NO: 1 or 2, whereby proliferation of the epidermal cells is induced.

17. The method of claim 16, wherein the peptide consists of the amino acid sequence of SEQ ID NO: 1.

18. The method of claim 16, wherein the peptide consists of the amino acid sequence of SEQ ID NO: 2.

19. The method of claim 16, wherein the epidermal cells are human epidermal keratinocytes (HEKs).

20. The method of claim 9, wherein the muscle cells are in a subject.

21. A method for reducing appearance of wrinkles on skin of a subject, comprising treating skin having appearance of wrinkles with an effective amount of a composition, wherein the composition comprises a peptide, wherein the peptide consists of an amino acid sequence of SEQ ID NO: 1 or 2, whereby the appearance of wrinkles on the skin is reduced.

22. The method of claim 21, wherein the peptide consists of the amino acid sequence of SEQ ID NO: 1.

23. The method of claim 21, wherein the peptide consists of the amino acid sequence of SEQ ID NO: 2.


SEQUENCE LISTING (80 sequences)

SEQ ID NO: 1 - protein, 4 aa, Artificial Sequence
  Synthetic
KKYK

SEQ ID NO: 2 - protein, 4 aa, Artificial Sequence
  Synthetic
KWKK

SEQ ID NO: 3 - protein, 4 aa, Artificial Sequence
  Synthetic
KYWL

SEQ ID NO: 4 - protein, 4 aa, Artificial Sequence
  Synthetic
KYWF

SEQ ID NO: 5 - protein, 4 aa, Artificial Sequence
  Synthetic
YPAK

SEQ ID NO: 6 - protein, 4 aa, Artificial Sequence
  Synthetic
NKKY

SEQ ID NO: 7 - protein, 4 aa, Artificial Sequence
  Synthetic
KYKI

SEQ ID NO: 8 - protein, 4 aa, Artificial Sequence
  Synthetic
NYKI

SEQ ID NO: 9 - protein, 4 aa, Artificial Sequence
  Synthetic
FCKK

SEQ ID NO: 10 - protein, 4 aa, Artificial Sequence
  Synthetic
YWFY

SEQ ID NO: 11 - protein, 5 aa, Artificial Sequence
  Synthetic
FYPAK

SEQ ID NO: 12 - protein, 4 aa, Artificial Sequence
  Synthetic
WFYP

SEQ ID NO: 13 - protein, 4 aa, Artificial Sequence
  Synthetic
YNKK

SEQ ID NO: 14 - protein, 5 aa, Artificial Sequence
  Synthetic
KALAI

SEQ ID NO: 15 - protein, 4 aa, Artificial Sequence
  Synthetic
YGCY

SEQ ID NO: 16 - protein, 5 aa, Artificial Sequence
  Synthetic
CCYKK

SEQ ID NO: 17 - protein, 5 aa, Artificial Sequence
  Synthetic
WFYPA

SEQ ID NO: 18 - protein, 4 aa, Artificial Sequence
  Synthetic
GKVF

SEQ ID NO: 19 - protein, 5 aa, Artificial Sequence
  Synthetic
NKYWF

SEQ ID NO: 20 - protein, 4 aa, Artificial Sequence
  Synthetic
KFFC

SEQ ID NO: 21 - protein, 5 aa, Artificial Sequence
  Synthetic
KYWFY

SEQ ID NO: 22 - protein, 4 aa, Artificial Sequence
  Synthetic
IPSP

SEQ ID NO: 23 - protein, 4 aa, Artificial Sequence
  Synthetic
KVFL

SEQ ID NO: 24 - protein, 5 aa, Artificial Sequence
  Synthetic
YWFYP

SEQ ID NO: 25 - protein, 5 aa, Artificial Sequence
  Synthetic
WKTYW

SEQ ID NO: 26 - protein, 4 aa, Artificial Sequence
  Synthetic
NRNY

SEQ ID NO: 27 - protein, 4 aa, Artificial Sequence
  Synthetic
FANL

SEQ ID NO: 28 - protein, 5 aa, Artificial Sequence
  Synthetic
FCKKP

SEQ ID NO: 29 - protein, 4 aa, Artificial Sequence
  Synthetic
GKIP

SEQ ID NO: 30 - protein, 4 aa, Artificial Sequence
  Synthetic
YLQK

SEQ ID NO: 31 - protein, 4 aa, Artificial Sequence
  Synthetic
WFYG

SEQ ID NO: 32 - protein, 4 aa, Artificial Sequence
  Synthetic
CYKK

SEQ ID NO: 33 - protein, 4 aa, Artificial Sequence
  Synthetic
YSSY

SEQ ID NO: 34 - protein, 4 aa, Artificial Sequence
  Synthetic
FYPA

SEQ ID NO: 35 - protein, 4 aa, Artificial Sequence
  Synthetic
TYNK

SEQ ID NO: 36 - protein, 4 aa, Artificial Sequence
  Synthetic
KKLT

SEQ ID NO: 37 - protein, 4 aa, Artificial Sequence
  Synthetic
CKKP

SEQ ID NO: 38 - protein, 4 aa, Artificial Sequence
  Synthetic
FFCK

SEQ ID NO: 39 - protein, 4 aa, Artificial Sequence
  Synthetic
YGAK

SEQ ID NO: 40 - protein, 4 aa, Artificial Sequence
  Synthetic
KNNY

SEQ ID NO: 41 - protein, 4 aa, Artificial Sequence
  Synthetic
KALA

SEQ ID NO: 42 - protein, 5 aa, Artificial Sequence
  Synthetic
KFFCK

SEQ ID NO: 43 - protein, 4 aa, Artificial Sequence
  Synthetic
RNYL

SEQ ID NO: 44 - protein, 4 aa, Artificial Sequence
  Synthetic
YLKP

SEQ ID NO: 45 - protein, 4 aa, Artificial Sequence
  Synthetic
QLGK

SEQ ID NO: 46 - protein, 4 aa, Artificial Sequence
  Synthetic
LKPF

SEQ ID NO: 47 - protein, 4 aa, Artificial Sequence
  Synthetic
KNYK

SEQ ID NO: 48 - protein, 4 aa, Artificial Sequence
  Synthetic
WKKK

SEQ ID NO: 49 - protein, 4 aa, Artificial Sequence
  Synthetic
KYWL

SEQ ID NO: 50 - protein, 4 aa, Artificial Sequence
  Synthetic
KKWK

SEQ ID NO: 51 - protein, 4 aa, Artificial Sequence
  Synthetic
KYWF

SEQ ID NO: 52 - protein, 4 aa, Artificial Sequence
  Synthetic
YPAK

SEQ ID NO: 53 - protein, 4 aa, Artificial Sequence
  Synthetic
NKKY

SEQ ID NO: 54 - protein, 4 aa, Artificial Sequence
  Synthetic
KYKI

SEQ ID NO: 55 - protein, 4 aa, Artificial Sequence
  Synthetic
NYKI

SEQ ID NO: 56 - protein, 4 aa, Artificial Sequence
  Synthetic
FCKK

SEQ ID NO: 57 - protein, 4 aa, Artificial Sequence
  Synthetic
KKKW

SEQ ID NO: 58 - protein, 4 aa, Artificial Sequence
  Synthetic
YWFY

SEQ ID NO: 59 - protein, 4 aa, Artificial Sequence
  Synthetic
KKYK

SEQ ID NO: 60 - protein, 4 aa, Artificial Sequence
  Synthetic
KFKK

SEQ ID NO: 61 - protein, 4 aa, Artificial Sequence
  Synthetic
KKKY

SEQ ID NO: 62 - protein, 4 aa, Artificial Sequence
  Synthetic
KYKK

SEQ ID NO: 63 - protein, 4 aa, Artificial Sequence
  Synthetic
YKKK

SEQ ID NO: 64 - protein, 4 aa, Artificial Sequence
  Synthetic
FKKK

SEQ ID NO: 65 - protein, 4 aa, Artificial Sequence
  Synthetic
KKKI

SEQ ID NO: 66 - protein, 4 aa, Artificial Sequence
  Synthetic
KKFK

SEQ ID NO: 67 - protein, 4 aa, Artificial Sequence
  Synthetic
KKIK

SEQ ID NO: 68 - protein, 4 aa, Artificial Sequence
  Synthetic
KKKF

SEQ ID NO: 69 - protein, 4 aa, Artificial Sequence
  Synthetic
KKLK

SEQ ID NO: 70 - protein, 4 aa, Artificial Sequence
  Synthetic
KLKK

SEQ ID NO: 71 - protein, 4 aa, Artificial Sequence
  Synthetic
KKKS

SEQ ID NO: 72 - protein, 4 aa, Artificial Sequence
  Synthetic
LKKK

SEQ ID NO: 73 - protein, 4 aa, Artificial Sequence
  Synthetic
IKKK

SEQ ID NO: 74 - protein, 4 aa, Artificial Sequence
  Synthetic
KIKK

SEQ ID NO: 75 - protein, 4 aa, Artificial Sequence
  Synthetic
KSKK

SEQ ID NO: 76 - protein, 4 aa, Artificial Sequence
  Synthetic
KKSK

SEQ ID NO: 77 - protein, 4 aa, Artificial Sequence
  Synthetic
KKPK

SEQ ID NO: 78 - protein, 4 aa, Artificial Sequence
  Synthetic
KKKL

SEQ ID NO: 79 - protein, 4 aa, Artificial Sequence
  Synthetic
KPKK

SEQ ID NO: 80 - protein, 4 aa, Artificial Sequence
  Synthetic
PKKK